(19) United States
(12) Patent Application Publication
(10) Pub. No.: US 2004/0197299 A1
(43) Pub. Date: Oct. 7, 2004

(54) Topically applicable cosmetic/dermatological compositions comprising hydrolase polypeptides having amidase activity and/or products modulating the activity thereof

(75) Inventors: Caroline Delattre, Paris (FR); Dominique Bernard, Paris (FR); Bruno Mehul, Opio (FR)

Correspondence Address: BURNS DOANE SWECKER & MATHIS L L P, POST OFFICE BOX 1404, ALEXANDRIA, VA, 22313-1404, US

(73) Assignee: SOCIETE L'OREAL, S.A., PARIS (FR)

(21) Appl. No.: 10/757,505

(22) Filed: Jan. 15, 2004

Related U.S. Application Data
(60) Provisional application No. 60/441,741, filed on Jan. 23, 2003.

(30) Foreign Application Priority Data
Jan. 16, 2003 (FR) 03/00454

(51) Int. Cl. A61K 38/46; A61K 31/74

(52) U.S. Cl. 424/78.02; 424/94.6

(57) ABSTRACT

Hydrolase polypeptides having amidase activity and products capable of modulating the activity thereof are well suited for the treatment of, inter alia, dry skins and/or the signs of skin aging and/or for combating skin desquamation.

### CROSS-REFERENCE TO PRIORITY/PROVISIONAL APPLICATIONS

This application claims priority under 35 U.S.C. § 119 of FR 03/00454, filed Jan. 16, 2003, and of provisional application Serial No. 60/441,741, filed Jan. 23, 2003, both hereby expressly incorporated by reference and both assigned to the assignee hereof. This application is also a continuation of said '741 provisional.

### BACKGROUND OF THE INVENTION

1. Technical Field of the Invention

The present invention relates to a composition comprising, in a physiologically acceptable medium suitable for topical application to the skin, at least one compound chosen from (i) a polypeptide of the family of hydrolases with amidase activity or a precursor thereof and (ii) a product capable of modulating the activity of the said-polypeptide; and their uses, in particular, for modulating the phenomenon of desquamation.

2. Description of Background and/or Related and/or Prior Art

Desquamation is a natural phenomenon linked to the fact that the epidermis, which constitutes the top layer of the skin, is in constant regeneration.

The epidermis consists of several strata of cells, of which the deepest is the basal stratum consisting of undifferentiated cells. Over time, these cells will differentiate and migrate to the surface of the epidermis, constituting the different strata thereof, including the formation, at the surface of the epidermis, of corneocytes which are dead cells which are eliminated by desquamation.

This surface loss is compensated by the migration of cells from the basal stratum to the surface of the epidermis: this constitutes the perpetual renewal of the skin. A forced elimination of the horny layer therefore accelerates renewal and makes it possible to combat aging.

At the same time, these cells continue their differentiation, of which the last stage is the corneocyte. They are in fact dead cells which constitute the last layer of the epidermis, that is to say the outermost layer also called stratum corneum.

Skin aging results from intrinsic or extrinsic factors which result in the appearance of wrinkles and fine lines, in yellowing of the skin which develops a wrinkled appearance accompanied by the appearance of pigmented spots, in the disorganization of the elastin and collagen fibers, causing a loss of elasticity, suppleness and firmness.

Some of these signs of aging are more particularly linked to intrinsic or physiological aging, that is to say to “normal” aging linked to age or chronobiological aging, whereas others are more specific to extrinsic aging, that is to say aging caused in general by the environment; it involves more particularly photoaging caused by exposure to the sun, to light or to any other radiation.

Various agents with desquamating properties, intended for combating skin aging, are known in the prior art.

Thus, U.S. Pat. No. 4,603,146 describes the use of retinoic acid and of its derivatives in cosmetic compositions, for combating skin aging.

Moreover, numerous commercial cosmetic compositions include α-hydroxy acids, such as lactic acid, glycolic acid or citric acid, for treating skin aging.

Finally, β-hydroxy acids, and more especially salicylic acid and its derivatives, are known for their desquamating properties (WO-A-93/10756 and U.S. Pat. No. 4,767,750).

All these compounds have an action against aging of the skin by promoting desquamation, that is to say the elimination of the “dead” cells present at the surface of the horny layer of the epidermis. This “desquamating” property is also called, often wrongly, keratolytic property.

However, the prior art compounds also have side effects, which consist of prickling, tightness, overheating and redness which are unpleasant for the user.

Serious need therefore remains to provide novel effective compounds which can be used in a composition suitable for topical application to the skin, for facilitating in particular desquamation of the skin.

### SUMMARY OF THE INVENTION

It has now surprisingly and unexpectedly been determined that aspartylglucosaminidase AGA (EC 3.5.1.26) belonging to the family of hydrolases with amidase activity (EC 3.5.1.X), is present in the epidermis in the stratum corneum and has a prodesquamating activity.

However, the study of certain mutations of AGA responsible for a serious genetic disease called aspartylglucosaminuria (AGU) had shown up until now an expression of AGA mainly in the hepatocytes, the pyramidal cells of the cerebral cortex, the proximal tubule cells of the kidney and, to a lesser degree, in the connective tissue (Enomaa N. E. et al., (1993), The Journal of Histochemistry and Cytochemistry, Vol 41, No. 7, pp 981-989; Arvio et al., (1999), J. Med Genet., 36, 398-404). No mention was made to any expression or activity of AGA, afortiori of a prodesquamating activity, in the epidermis, in particular in the stratum corneum.

The present invention therefore features compositions comprising, in a physiologically acceptable medium suitable for topical application to the skin, at least one compound chosen from (i) a polypeptide of the family of hydrolases, with amidase activity or a precursor thereof and (ii) a product capable of modulating the activity of the said polypeptide.

This invention also features the use of the said compounds or of the said compositions in a regime or regimen for modulating, in particular for promoting, desquamation and/or for modulating cell renewal of the epidermis and/or for modulating the hydration of the skin and/or for modulating cell proliferation and/or differentiation in the skin and/or for facilitating the skin penetration of active agents of cosmetic and/or dermatological compositions and/or for combating bacterial adhesion.

### DETAILED DESCRIPTION OF BEST MODE AND SPECIFIC/PREFERRED EMBODIMENTS OF THE INVENTION

More particularly according to the present invention, in a first embodiment thereof, the compositions contain, as active ingredient, at least (i) one polypeptide of the family of hydrolases with amidase activity or a precursor thereof.

Hydrolases with amidase activity (EC 3.5.1.X) act on carbon/nitrogen bonds other than peptide bonds. In particular, aspartylglucosaminidase AGA (EC 3.5.1.26) is a lysosomal enzyme which hydrolyses the N-acetylglucosamine-asparagine bonds of glycopeptides and human glycoproteins (McGovern M M et al., (1983), The Journal of Biological Chemistry, 258, 17, 10743-10747).

By way of example of compounds of the family of hydrolases with enzymatic activity, there may be mentioned the compounds EC 3.5.1.1 to EC 3.5.1.89 of the conventional nomenclature, among which asparaginase, glutaminase, amidase, urease, aminoacylase, aspartoacylase, ceramidase, peptidyl-glutaminase, formamidase, pentanamidase, and aspartylglucosaminidase AGA.

A polypeptide with aspartylglucosaminidase activity or a precursor thereof will be preferably employed in the context of the invention.

The expression “precursor of a polypeptide” is understood to mean a nonactive or “pro-” form of the enzyme which, after conversion, leads to the active form of the enzyme. This conversion may consist in a separation of the signal peptide and an intramolecular autoproteolysis.

In the context of the invention, the said polypeptide with aspartylglucosaminidase activity may be chosen from:

a) a polypeptide of human origin or a homologue of the said polypeptide having an aspartylglucosaminidase activity;

b) an enzymatic or biomimetic analogue of the said polypeptide according to a), having an aspartylglucosaminidase activity;

c) a fragment of the said polypeptide according to a), the said fragment having an aspartylglucosaminidase activity;

d) a polypeptide or an enzymatic analogue or fragment of polypeptide according to a), b) or c) in an active form of the heterodimer or heterotetramer type;

e) a polypeptide or an enzymatic analogue or a polypeptide fragment according to a), b) or c) having undergone one or more modifications.

The polypeptide of human origin having an aspartylglucosaminidase activity comprises, for example, the peptide sequence described under SwissProt Accession No. P20933 (Fisher et al., 1990, FEBS Letter, 269(2), 440-444) or its homologues.

In general, the expression “homologue” of a polypeptide or of a peptide sequence is understood to mean any polypeptide or any peptide sequence which is identical to at least 50%, preferably to at least 80% and still more preferably to at least 95% of a polypeptide or of a defined polypeptide sequence, in the same species or in a different species; in the latter case, it is also designated “orthologous polypeptide”.

As polypeptides with aspartylglucosaminidase activity which can be used according to the invention, there may be mentioned, for example, the polypeptides of known AGA sequences in eukaryotes, in particular the AGA sequences of mammals (mice, humans), of yeast and of plants and their orthologues (homologous polypeptide, in a different species), in particular:

| | SwissProt |
|---|---|
| Species | Accession No. |
| Caenorhabditis elegans | (Q21697) |
| (nematode) | |
| Flavobacterium meningosepticum | (Q47898) |
| (bacterium) | |
| Homo sapiens (human) | (P20933) |
| Mus musculus (mouse) | (Q64191) |
| Sus scrofa (pig) | (P30918) |
| Rattus norvegicus (rat) | (P30919) |
| Spodoptera frugiperda (insect) | (O02467) |

The expression “percentage identity” between two peptide sequences or amino acid sequences is understood to mean a percentage of amino acid residues which are identical between the two sequences to be compared, obtained after the best alignment, that is to say the optimum alignment obtained, for example, by means of the Smith and Waterman local homology algorithm (1981, Ad. App. Math., 2:482), by means of the Neddleman and Wunsch local homology algorithm (1970, J.Mol. Biol., 48: 443), by means of the Pearson and Lipman search for similarity method (1988, Proc. Natl. Acad. Sci. USA, 85:2444), by means of computer software packages using these algorithms (GAP, BESTFIT, BLAST P, BLAST N available at the NCBI site, FASTA and TFASTA in the Wisconsin Genetics Software Package, Genetics Computer Group, 575 Science Dr, Madison, Wis.).

The expression “enzymatic or biomimetic analogues” having an aspartylglucosaminidase activity is understood to mean any molecule capable of hydrolyzing the carbon-nitrogen bonds other than the peptide bonds, in particular N-acetylglucosamine-asparagine bonds of glycopeptides and human glycoproteins. There may be mentioned, as examples of enzymatic analogues or “synzymes”:

artificial enzymes which have the capacity to catalyse reactions by binding to transition states of the substrate; as hydrolase-like enzymes, for example, there may be mentioned cyclodextrins, cyclophanes, cyclic porphyrins;

catalytic antibodies or “abzymes”, which selectively bind to a transition state analogue of the substrate (Schultz PG et al., 1986, Science, 234, 1570-1573), and which can henceforth be obtained by immunization in vitro using the stable analogue of the transition state as antigen;

RNA enzymes or “ribozymes” which have a catalytic power as described in Bartel DP et al., 1993, Science, 251:1411-1418.

The expression “polypeptide fragment” is understood to mean any fragment characterized in that its size makes it possible to reconstitute the active site of the enzyme necessary for the activity and the specificity of aspartylglucosaminidase. The amino acid Threonine 206 is described as being necessary for the activity of aspartylglucosaminidase as well as the combination of its two alpha and beta chains into a heterodimer. Polypeptides having a size between 1 and 50 kD which preserve this amino acid 206 will therefore be chosen. This polypeptide fragment may be obtained by proteolysis or synthetically according to known methods.

The enzymatic polypeptide or analogue or polypeptide fragment with aspartylglucosaminidase activity according to the invention may be in the form of a precursor or in the active form of a heterodimer or heterotetramer.

The aspartylglucosaminidase precursor corresponds to the non-active form of the enzyme which, after separation of the signal peptide and intramolecular autoproteolysis, leads to two subunits of 24 kDa (Alpha) and 18 kDa (Beta) whose combination in the form of a heterodimer or heterotetramer leads to the active form of the enzyme (Saarela J., 1998, The Journal of Biochemistry, Vol. 273, No. 39, 25320-25328).

It is also possible to use in the context of the invention an enzymatic polypeptide or analogue or polypeptide fragment with aspartylglucosaminidase activity according to the invention which has undergone one or more modifications. The expression “modification” is understood to mean any substitution, deletion and/or insertion of an amino acid or of a reduced number of amino acids, in particular by substitution of natural amino acids with unnatural amino acids or pseudoamino acids at positions such that the modifications do not significantly impair the biological activity of the AGA. The modification may also correspond to conservative substitutions, that is to say substitutions of amino acids of the same class, such as substitutions of amino acids with uncharged side chains (asparagine, glutamine, serine, threonine, tyrosine), of amino acids with basic side chains (lysine, arginine and histidine), of amino acids with acid side chains (aspartic acid and glutamic acid); of amino acids with apolar side chains (glycine, alanine, valine, leucine, isoleucine, proline, phenylalanine, methionine, tryptophan and cysteine).

Preferably, the polypeptide of human origin having an aspartylglucosaminidase activity can be isolated and purified from the stratum corneum of the human epidermis; it has an apparent molecular mass between 10 and 50 kD, particularly between 15 and 48 kD and in particular between 15 and 24 kD. It may be obtained according to known methods of extraction and purification, and in particular according to the protocol described in Example 1 below, comprising the successive steps of extraction in the presence of EDTA, of filtration, of cationic chromatography and of activity assay.

The polypeptide belonging to the family of hydrolases with amidase activity, and in particular AGA, may be of natural or synthetic, in particular recombinant, origin.

The expression “natural origin” is understood to mean a polypeptide in the pure state or in solution at various concentrations, which is obtained by various methods of extraction from a tissue (skin, liver and the like) of natural origin, in particular the stratum corneum of the human epidermis.

The expression “synthetic origin” is understood to mean a polypeptide in the pure state or in solution at various concentrations, which is obtained chemically or by production in an organism after introduction into this organism of the elements necessary for this production.

In a composition according to the invention, the polypeptide of the family of hydrolases with amidase activity is generally in a quantity between 10⁻⁶ and 5% by total weight of the composition, preferably between 10⁻⁴ and 1% by total weight of the composition and more preferably between 0.001% and 0.1% by total weight of the composition.

According to a first variant, the AGA polypeptide is kept in the form of a non-active precursor in the composition up to its application to the skin, where the pH and ionic strength conditions are favorable to the autoproteolysis reaction which is necessary for the activation of the AGA. It is known that the activation of the AGA is promoted and/or triggered at a pH between 5 and 8.

According to a second variant, the polypeptide AGA is in its active form in the composition, the said composition already containing the elements necessary for the autoproteolysis of the precursor form (favorable physiological conditions of pH and ionic strength). The said elements promoting the AGA activity may be present in the same compartment as the AGA polypeptide or in a separate compartment, the activation of the AGA being deferred until the application of the said composition, as described in detail below (preferred mode).

According to a second embodiment, the composition comprises, as active ingredient, at least (ii) one product capable of modulating the activity of the polypeptide of the family of hydrolases with amidase activity, in particular of AGA.

The expression “modulating the activity of the polypeptide” is understood to mean modulating the expression of the said polypeptide and/or modulating the enzymatic activity of the said polypeptide. The expression product capable of modulating the activity of the polypeptide of the family of hydrolases with amidase activity should therefore be understood to mean any element capable of increasing or decreasing the quantity of the said polypeptide, either by stimulating or by repressing the synthesis of the said polypeptide, whether by a transcriptional, post-transcriptional, translational or post-translational mechanism, or by stimulating or repressing the catabolism of the said polypeptide, or by stimulating or by repressing the intrinsic activity of the said polypeptide.

These products may be selected according to a screening method based on the measurement of a variation of the expression and/or of the activity of the said polypeptide.

By way of examples of methods well known to a person skilled in the art, there may be mentioned:

quantitative RT-PCR type tests as described in White B. A., (1997), Methods of Molecular Biology, Humana Press, Vol 67;

enzymatic tests such as that described in Mononen I. T. et al., (1993), Analytical Biochemistry, 208, 372-374;

ELISA tests with specific antibodies as described in Kemeny D. M., (1991), A Practical Guide to ELISA, Pergamon Press.

This product capable of modulating the activity of the said polypeptide may be an activator or an inhibitor.

The expression “activator” is understood to mean either a product, or a set of products, capable of stimulating the activity of the said polypeptide, for example of increasing the rate of the enzymatic reaction, measured by the increase in the quantity of substrates digested per unit of time during the bringing of the AGA polypeptide into contact with the activator.

In particular, these products can act by modifying the environment of the enzyme, in particular the pH and ionic strength conditions so as to make them favorable to the activation of the said enzyme.

This bringing into contact may take place at the time of the application of the composition containing the activator to the epidermis of the skin which contains, in particular in the stratum corneum, endogenous AGA polypeptides.

By way of example of activator, it is possible to use Sodium Dodecyl Sulphate (SDS) at non-irritating concentrations of 1% to 5% for rinse-out products or preferably Sodium Lauryl Ether Sulphate at non-irritating concentrations of between 10% and 25% for the rinse-out products or between 0.001% and 0.1% for the care products.

These activators generally represent between 0.01% and 50% of the total weight of the composition, preferably between 0.1% and 1% of the total weight of the composition. The highest concentrations are reserved for applications of the “peeling” type. For the other cosmetic applications, proportions of less than 10%, in particular of less than 1%, will be preferably used.

The expression “inhibitor” is understood to mean a product capable of inhibiting the activity of the said polypeptide, that is to say of decreasing the rate of enzymatic reaction, measured for example by the decrease in the quantity of substrates digested per unit of time when the said product is brought into contact with the polypeptide. In particular, these products can act at the level of the active site of the enzyme or by competition in relation to binding to the substrate.

This bringing into contact may take place at the moment of application of the composition containing the inhibitor to the epidermis of the skin which contains, in particular in the stratum corneum, endogenous AGA polypeptides.

As examples of inhibitors, there may be mentioned L-asparagine which reversibly modulates AGA and its analogue 5-diaxo-4-oxo-L-norvaline, which reversibly modulates AGA (Noronski et al., FEBS Letters, 412, 149-152, 1997). There may also be mentioned N-acetylcysteine, asparagine, aspartylalanine, aspartylcyclohexylamine and p-chloromercuribenzoic acid.

These inhibitors generally represent between 10⁻⁶ and 5% of the total weight of the composition, preferably between 0.1% and 1% of the total weight of the composition.

These inhibitors are particularly advantageous since they make it possible to increase the barrier effect of the skin by promoting the development of the thickness of the stratum corneum and thus to contribute to:

combating the harmful effects of UV radiation involved in the photoaging process;

and/or combating the thinning of the skin which is one of the signs of chronobiological aging.

The use of the inhibitors is particularly recommended in the treatment of sensitive and/or aged skins and in the treatment of atopic dermatites linked to barrier function disorders.

According to a preferred embodiment, the composition according to the invention comprises, in a physiologically acceptable medium suitable for topical application to the skin, at least (i) one polypeptide of the family of hydrolases with amidase activity or a precursor thereof and (ii) an activator of the said polypeptide.

Such a combination makes it possible to decrease the active concentration of the said active agents (activator product) because of the additive effects. It is thus possible to obtain a less irritating and less toxic composition, and a composition which is more effective than those of the prior art using only these active agents (activator product). This combination is particularly advantageous for activators such as SDS for example, which can exhibit, at a high concentration, an irritating effect on the skin.

The compounds (i) and (ii) may be administered together in the same composition, or separately in separate compositions, simultaneously or spaced out over time.

Advantageously, the polypeptide of the family of hydrolases with amidase activity and the activator of the said polypeptide are packaged so as not to be in contact with each other as described by the applicant in FR-2,780,645, for example in two different compositions, which may be either mixed at the time of application or applied successively or spaced out over time.

For example, the polypeptide of the family of hydrolases with amidase activity and the activator of the said polypeptide are packaged in separate compartments or separate phases. Such two-compartment packaging devices are, for example, described in FR-A-2-045,559, FR-A-2-105,332, FR-A-2-258,319, FR-A-2-293,375, FR-A-2-586,913 or FR-A-2-643,615.

It is also possible to prepare one of the compositions in an encapsulated form and/or in the form of microcapsules or microgranules immersed in the other composition, the microcapsules or microgranules being crushed at the time of application by rubbing on the skin, which thus allows mixing of the said polypeptide of the family of hydrolases with amidase activity with the activator of the said polypeptide.

According to one variant, the AGA polypeptide is in the form of a non-active precursor contained in microcapsules or microgranules immersed in the other composition comprising the elements necessary for the activation of the AGA precursor; this activation is deferred until the application of the composition to the skin.

The invention also relates to a composition comprising at least one compound chosen from (i) a polypeptide of the family of hydrolases with amidase activity or a precursor thereof and (ii) an activator of the said polypeptide, characterized in that it additionally comprises at least one other desquamating agent.

The expression “desquamating agent” is understood to mean any compound capable of acting:

either on desquamation by promoting exfoliation;

or on the enzymes involved in the desquamation or the degradation of the corneodesmosomes.

There may be mentioned for example: β-hydroxy acids, in particular salicylic acid and its derivatives such as 5-(n-octanoyl)salicylic acid; α-hydroxy acids, such as glycolic, citric, lactic, tartaric, malic or mandelic acids; α- or β-keto acids; urea; gentisic acid; oligofucoses; cinnamic acid; extract of Saphora japonica; resveratrol and its derivatives; glycosidases; stratum corneum chymotryptic enzyme (SCCE) or even other serine or cysteine proteases (trypsin or chymotrypsin-like or cysteine protease as described by the applicant in FR-2-767,833); mineral salt-chelating agents: EDTA; N-acyl-N,N′,N′-ethylenediaminetriacetic acid; aminosulphonic compounds and in particular (N-2-hydroxyethylpiperazine-N-2-ethane)sulphonic acid (HEPES); 2-oxothiazolidine-4-carboxylic acid (procysteine) derivatives; glycine-type alpha-amino acid derivatives (as described in EP-0-852,949, and sodium methylglycine diacetate marketed by BASF under the trademark TRILON M®); calcium chelators; honey; sugar derivatives such as O-octanoyl-6-D-maltose and N-acetylglucosamine; reducing agents, glutathione, cysteine; or certain carbohydrates such as those defined in WO-A-97/12597); jasmonic acid and its derivatives; retinoids such as retinoic acid (all-trans or 13-cis) and its derivatives, retinol (vitamin A) and its esters such as retinol palmitate, retinol acetate and retinol propionate and their salts, or retinal.

By way of example, the other desquamating agent may be introduced into the compositions used according to the invention in a quantity representing from 0.01% to 50% by total weight of the composition and better still from 0.1% to 3%.

Such a combination with a polypeptide of the family of hydrolases with amidase activity makes it possible to decrease the active concentration of the other products with desquamating activity because of the additive effects. It is thus possible to obtain a less irritating and less toxic composition, and a composition which is more effective than those of the prior art using only these active agents.

The invention also relates to a composition comprising at least one compound chosen from (i) a polypeptide of the family of hydrolases with amidase activity or a precursor thereof and (ii) a product capable of modulating the activity of the polypeptide, characterized in that it additionally comprises at least one compound chosen from moisturizing agents; propigmenting agents; agents stimulating the synthesis of dermal or epidermal macromolecules and/or preventing their degradation; agents stimulating the proliferation or differentiation of keratinocytes; relaxing agents; antipollution and/or anti-free radical agents; UV-screening agents; permeating agents; cicatrizing agents; and mixtures thereof.

In a composition according to the invention, the moisturizing agents and/or the agents stimulating the proliferation of the keratinocytes and/or the cicatrizing agents will be preferably combined with the compounds chosen from (i) a polypeptide of the family of hydrolases with amidase activity or a precursor thereof and (ii) an activator of the said polypeptide.

The agents stimulating the synthesis of dermal or epidermal macromolecules and/or preventing their degradation; the agents stimulating the differentiation of the keratinocytes, the antipollution and/or anti-free radical agents; the UV-screening agents; the permeating agents or mixtures thereof will be preferably combined, in a composition according to the invention, with an inhibitor of the polypeptide of the family of hydrolases with amidase activity.

The expression “moisturizing agent” is understood to mean:

either a compound acting on the barrier function, in order to maintain the hydration of the stratum corneum, or an occlusive compound. There may be mentioned ceramides, sphingoid-based compounds, lecithins, glycosphingolipids, phospholipids, cholesterol and its derivatives, phytosterols (stigmasterol, β-sitosterol, campesterol), essential fatty acids, 1,2-diacylglycerol, 4-chromanone, pentacyclic triterpenes such as ursolic acid, petroleum jelly and lanolin;

or a compound which directly increases the water content of the stratum corneum, such as threalose and its derivatives, hyaluronic acid and its derivatives, glycerol, pentanediol, sodium pidolate, serine, xylitol, sodium lactate, glycerol polyacrylate, ectoine and its derivatives, chitosan, oligo- and polysaccharides, cyclic carbonates, N-lauroylpyrrolidonecarboxylic acid, and N-(α-benzoyl-L-arginine;

or a compound which activates the sweat glands, such as caffeic acid or a compound which promotes sweating by local vasodilation.

As “propigmenting agent”, there may be mentioned the extract of burnet (Sanguisorba officinalis) marketed by MARUZEN and the extracts of chrysanthemum (Chrysanthemum morifolium).

The “agents stimulating the proliferation of the keratinocytes” comprise in particular retinoids such as retinol and its esters, including retinyl palmitate; phloroglucinol; the extracts of nut cake which are marketed by GATTEFOSSE; and the Solanum tuberosum extracts marketed by SEDERMA.

The “agents stimulating the differentiation of the keratinocytes” comprise for example minerals such as calcium; lupin extract marketed by SILAB under the trademark Photopreventine®; sodium beta-sitosteryl sulphate marketed by SEPORGA under the trademark Phytocohesine®; and the maize extract marketed by SOLABIA under the trademark Phytovityl®.

The “relaxing agents” comprise in particular alverine and its salts, manganese and its salts; magnesium and its salts; sapogenins such as diosgenin and natural extracts containing them (such as the extracts of Wild Yam), and the hexapeptide Argireline® marketed by LIPOTEC and its salts.

The expression “antipollution agent” is understood to mean any compound capable of trapping ozone, mono- or polycyclic aromatic compounds such as benzopyrene and/or heavy metals such as cobalt, mercury, cadmium and/or nickel.

The expression “anti-free radical agent” is understood to mean any compound capable of trapping free radicals such as vitamin E and its derivatives; bioflavonoids; coenzyme Q10 or ubiquinone; certain enzymes such as catalase, superoxide dismutase, lactoperoxidase, glutathione peroxidase and quinone reductases or their mimetics; glutathione; benzylidenecamphor; benzylcyclanones; substituted naphthalenones; pidolates; phytantriol; gamma-oryzanol; lignans; and melatonin.

The composition according to the invention may also contain “UVA and/or UVB screening agents” in the form of organic or inorganic compounds, the latter being optionally coated in order to make them hydrophobic.

The organic screening agents may be chosen in particular from: anthranilates, in particular menthyl anthranilate; benzophenones, in particular benzophenone-1, benzophenone-3, benzophenone-5, benzophenone-6, benzophenone-8, benzophenone-9, benzophenone-12 and preferably benzophenone-3 (Oxybenzone), or benzophenone-4 (Uvinul MS40 available from B.A.S.F.); benzylidenecamphors, in particular 3-benzylidenecamphor, benzylidenecamphorsulphonic acid, camphor benzalkoniummethosulphate, polyacrylamidomethylbenzylidenecamphor, terephthalylidenedicamphorsulphonic acid, and preferably 4-methylbenzylidenecamphor (Eusolex 6300 available from Merck); benzimidazoles, in particular benzimidazilate (Neo Heliopan AP available from Haarmann and Reimer), or phenylbenzimidazolesulphonic acid (Eusolex 232 available from Merck); benzotriazoles, in particular drometrizoletrisiloxane, or methylenebisbenzotriazolyltetramethylbutylphenol (Tinosorb M available from Ciba); cinnamates, in particular cinoxate, DEA methoxycinnamate, diisopropyl methylcinnamate, glyceryl ethylhexanoate dimethoxycinnamate, isopropyl methoxycinnamate, isoamyl cinnamate, and preferably ethocrylene (Uvinul N35 available from B.A.S.F.), octyl methoxycinnamate (Parsol MCX available from Hoffmann La Roche), or octocrylene (Uvinul 539 available from B.A.S.F.); dibenzoylmethanes, in particular butyl methoxydibenzoylmethane (Parsol 1789); imidazolines, in particular ethylhexyl dimethoxybenzylidene dioxoimidazoline; PABAs, in particular ethyl dihydroxypropyl PABA, ethylhexyldimethyl PABA, glyceryl PABA, PABA, PEG-25 PABA, and preferably diethylhexylbutamidotriazone (Uvasorb HEB available from 3V Sigma), ethylhexyltriazone (Uvinul T150 available from B.A.S.F.), or ethyl PABA (benzocaine); salicylates, in particular dipropylene glycol salicylate, ethylhexyl salicylate, homosalate, or TEA salicylate; triazines, in particular anisotriazine (Tinosorb S available from Ciba); drometrizole trisiloxane.

The inorganic screening agents preferably consist of zinc oxide and/or titanium dioxide, preferably of nanometric size, optionally coated with alumina and/or stearic acid.

“Pro-penetrating or permeating agents” may be advantageously added in order to reinforce the penetration of the active agents. There may be mentioned for example binary systems (U.S. Pat. No. 4,537,776) or solvents such as DMSO (U.S. Pat. No. 3,551,554) as vehicles, or active permeating agents which exist both in the free base form and in the acid addition salt form as described in EP-0-321,870 B1.

It is also possible to use “cicatrizing agents”, that is to say agents which promote cicatrization, in particular skin cicatrization, by promoting reepidermization (reepithelialization and normalization of the epidermis and of the dermis) and/or by limiting the phenomenon of retraction of the wound. There may be mentioned in particular inhibitors of the activity of retinoic acid described by the applicant in FR-2-753,627.

The compositions according to the invention contain a cosmetically or dermatologically acceptable medium, that is to say a medium which is compatible with the skin, the nails, the mucous membranes, the tissues and the hair. According to a preferred embodiment of the invention, the composition has a pH allowing optimum activity of the polypeptides used and preferably close to that of the skin, between 5 and 8.

In a known manner, the compositions according to the invention may also contain customary adjuvants in the cosmetic and dermatological fields, such as hydrophilic or lipophilic gelling agents, hydrophilic or lipophilic active agents, preservatives, antioxidants, solvents, perfumes, fillers and coloring matter. The quantities of these various adjuvants are those conventionally used in the fields considered, and are for example from 0.01% to 20% of the total weight of the composition. Of course, persons skilled in the art will be careful to choose this or these possible additives and/or their quantities such that the advantageous properties intrinsically attached to the composition in accordance with the invention, in particular the enzymatic activities of the polypeptides according to the invention, are not, or not substantially, impaired by the addition(s) envisaged.

As oils which can be used in the invention, there may be mentioned mineral oils (liquid paraffin), vegetable oils (karite oil, sweet almond oil), animal oils, synthetic oils, silicone oils (cyclomethicone) and fluorinated oils (perfluoropolyethers). It is also possible to use, as fat, fatty alcohols, fatty acids (stearic acid), waxes (paraffin, carnauba, beeswax).

As emulsifiers which can be used in the invention, there may be mentioned Polysorbate 60 and sorbitan stearate which are sold respectively under the trademarks Tween 60 and Span 60 by ICI. It is also possible to add thereto coemulsifiers such as PPG-3 myristyl ether sold under the trademark Emcol 249-3K by Witco.

As solvents which can be used in the invention, there may be mentioned low alcohols, in particular ethanol and isopropanol, propylene glycol.

As hydrophilic gelling agents, there may be mentioned carboxyvinyl polymers (carbomer), acrylic copolymers such as acrylate/alkyl acrylate copolymers, polyacrylamides, polysaccharides such as hydroxypropylcellulose, natural gums (xanthan) and clays, and, as lipophilic gelling agents, there may be mentioned modified clays such as bentones, fatty acid metal sols such as aluminum stearates, hydrophobic silica, polyethylenes and ethyl cellulose.

As hydrophilic active agents, it is possible to use proteins or protein hydrolysates, amino acids, polyols, urea, allantoin, sugars and sugar derivatives, water-soluble vitamins, starch, bacterial or plant, in particular Aloe vera, extracts.

As lipophilic active agents, it is possible to use tocopherol (vitamin E) and its derivatives, essential fatty acids, ceramides, essential oils.

The cosmetic compositions according to the invention are preferably provided in a form appropriate for administration by the topical route and to contain enzyme-type active agents such as the polypeptide of the family of hydrolases with amidase activity according to the invention, which exhibit instability in aqueous medium. The polypeptide is therefore preferably in a stabilized form.

The compositions are provided, in particular, in the form of aqueous-alcoholic or oily solutions or of lotion- or serum-type dispersions, of anhydrous or oily gels, of emulsions having a liquid or semi-liquid consistency of the milk type, which are obtained by dispersing a fatty phase in an aqueous phase (O/W) or conversely (W/O), of suspensions or emulsions having a soft, semi-solid or solid consistency of the cream or gel type, of microemulsions, or of microcapsules, microparticles or vesicular dispersions of the ionic and/or nonionic type. These compositions are prepared according to the customary methods. A preferred form which is especially suitable for compositions comprising enzyme active agents is a form of the W/O/W type as described in application EP-0-779,071.

The polypeptides of the family of hydrolases with amidase activity may also be in an immobilized form on polymeric supports as described in DE-1-8-824,072 or in microcapsules. It is possible to use in particular spheres of poly-beta-alanine (covalent bond) or liposomes/niosomes (compartments). Another solution consists in incorporating them into an anhydrous liquid medium (U.S. Pat. No. 5,322,683 A). It is also possible to use surfactants for the stabilization of the said polypeptides in an aqueous medium, or polyols associated with a structuring agent as described in FR-2-737,115.

The compositions may be provided in the form of a lotion, a cream, a milk, a gel or of foams for care of the skin, the mucous membranes and/or the keratinous fibers, or for cleansing the skin, of a mask, of microspheres or nanospheres or of vesicular dispersions consisting of ionic lipids (liposomes) and/or nonionic lipids. The compositions may also consist of solid preparations constituting cleansing soaps or cakes.

This invention also features a regime or regimen for the cosmetic treatment of dry skins, intended for combating skin disorders linked to desquamation and/or cell renewal of the epidermis and/or to hydration of the skin and/or to cell proliferation and/or differentiation in the skin, characterized in that a composition comprising at least one compound chosen from (i) a polypeptide of the family of hydrolases with amidase activity or a precursor thereof and (ii) an activator of the said polypeptide are applied to the skin, the mucous membranes and/or the keratinous fibers.

This method is intended in particular:

to promote desquamation, in particular by hydrolysis of the glycoproteins in the corneodesmosomes;

and/or to promote hydration of the skin and the osmotic status of the epidermis, in particular by releasing low-molecular-weight sugar chains;

and/or to promote cell renewal and/or cell proliferation and/or differentiation in the skin, in particular by releasing epidermal growth factors, such as amphiregulins or the epidermal growth factor (EGF).

The invention also relates to a method for the cosmetic treatment of sensitive skins and/or of aged skins, intended to increase the barrier effect of the skin, characterized in that a composition comprising at least one inhibitor of the polypeptide of the family of hydrolases with amidase activity is applied to the skin, the mucous membranes and/or the keratinous fibers.

These methods entail the application of the compositions of the invention according to the usual technique for using these compositions. For example: application of creams, gels, sera, ointments, lotions, milks to the skin, the scalp, the nails and/or the mucous membranes.

The invention also relates to the use of a composition comprising at least one compound chosen from (i) a polypeptide of the family of hydrolases with amidase activity or a precursor thereof and (ii) an activator of the said polypeptide for the preparation of a pharmaceutical composition intended for treating desquamation disorders, in particular hyperkeratosis, xerosis, ichtyosis, psoriasis or reactive keratosis.

The invention also relates to the use of a composition comprising at least one compound chosen from (i) a polypeptide of the family of hydrolases with amidase activity or a precursor thereof and (ii) an activator of the said polypeptide for the preparation of a pharmaceutical composition intended for promoting cicatrization, because of the fact that the degradation of the proteoglycans of the cellular matrix induces a recolonization of the cellular spaces by the keratinocytes.

Also falling within the scope of the invention is the use of a composition comprising at least one inhibitor of the polypeptide of the family of hydrolases with amidase activity, for the preparation of a pharmaceutical composition intended for treating atopic dermatitis.

The invention also relates to the use, in a cosmetic composition for topical application, of at least one compound chosen from (i) a polypeptide of the family of hydrolases with amidase activity or a precursor thereof and (ii) an activator of the said polypeptide, the said compounds or the said composition being intended to promote the desquamation and/or the hydration of the skin and/or cell renewal in the skin and/or cell proliferation and/or differentiation in the skin.

Also within the scope of the invention is the use, in a cosmetic composition for topical application, of at least one compound chosen from (i) a polypeptide of the family of hydrolases with amidase activity or a precursor thereof and (ii) an activator of the said polypeptide, the said compounds or the said composition being intended to facilitate skin penetration of active agents of cosmetic and/or dermatological compositions; the desquamation having the effect of decreasing the impermeability of the stratum corneum which normally forms an obstacle to the passage of products through the skin; the stratum corneum layer indeed plays a role of barrier, which is essential for the body, because it prevents, on the one hand, the entry of pathogens and other toxic products and, on the other hand, the outflow of physiological fluids.

The invention also relates to the use, in a cosmetic composition for topical application, of at least one compound chosen from (i) a polypeptide of the family of hydrolases with amidase activity or a precursor thereof and (ii) an activator of the said polypeptide, the said compounds or the said composition being intended to combat bacterial adhesion, in particular by releasing low-molecular-weight sugar chains and therefore by modifying, at the level of the corneocytes, the sites of bacterial adhesion and colonization.

That which follows presents the demonstration of the presence and of the prodesquamating activity of AGA in the stratum corneum. It moreover presents examples of formulation of compositions according to the invention without restricting the scope of the invention.

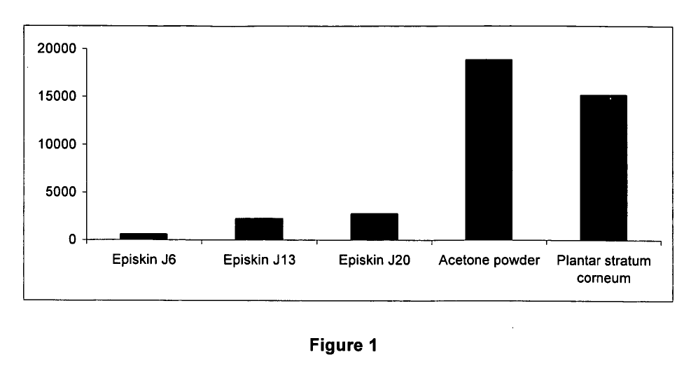

FIG. 1 is a histogram presenting the assay of the AGA activity with the substrate Asp-AMC on reconstructed skin Episkin®, acetone powder or plantar stratum corneum.

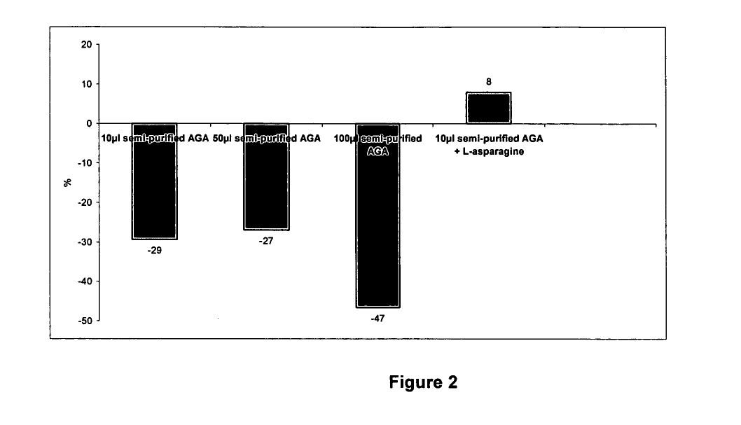

FIG. 2 is a histogram presenting the variation of the corneodesmosin signal after incubation of various quantities of semi-purified AGA with an acetone powder for 8 hours, control incubation buffer subtracted.

### EXAMPLES

### Example 1

### Demonstration of the Presence of an AGA Activity in the Stratum Corneum SC

a) Purification and Identification of AGA from the SC:

An SC extract is obtained by “scraping” the anterior surface of the leg with an extraction buffer at pH 4 containing 50 mM sodium acetate, 5 mM EDTA (ethylenediaminetetraacetate) and 0.1% Tween 20. After a series of filtrations, the extract is separated by cationic chromatography; the elution is carried out with a linear NaCl gradient in the same extraction buffer.

The purification of AGA is monitored by assaying of activity with the fluorescent substrate L-aspartic acid beta(7-amido-4-methylcoumarin) (Asp-AMC) according to the method described by Mononen I. T. et al., 1993, Analytical Biochemistry, 208, 372-374. 10 μl of each eluted fraction are incubated for 24 h at 37° C. with 100 μl of Asp-AMC substrate diluted to 1 mM in 0.1 M Tris-HCl buffer, pH 8. The fluorescence is quantified on a Biolumin reader (excitation/emission: 340/450 nm).

All the fractions having an AGA type activity (fractions 14 to 43) are then pooled and concentrated. A change of buffer is carried out on an EconoPac10DG column (Bio-Rad) with buffer at pH 7 containing 50 mM sodium phosphate, 5 mM EDTA, 150 mM NaCl and 0.1% Tween 20. A chromatographic separation of the gel filtration type (G200) is carried out and the AGA activity of each fraction is assayed with the substrate Asp-AMC.

A semi-purified AGA extract is obtained by pooling the fractions with high AGA activity (fractions 57 to 71).

It is then concentrated and analyzed on gel after SDS-PAGE separation (12% gel) and staining with silver nitrate.

Four major bands having the respective molecular weights 48, 24, 16 and 15 kDa were observed. Each band was analyzed by MALDI-TOF-type mass spectrometry after trypsin digestion. Analysis by peptide mapping in the NCBI data banks using the PROFOUND® software led to the identification of the A chain of AGA (band of 24 kDa with a recovery rate of 28% and a probability of 35%) and of the B chain of AGA (bands of 16 and 15 kDa with a respective recovery rate of 64% and 38% and a probability of 100% and 11%).

An Edman-type chemical sequencing confirmed the identification of AGA for these same bands.

In particular, the polypeptide isolated from the stratum corneum has a peptide sequence containing at least the succession of amino acids No. 136-150 of the human peptide sequence under the reference number SwissProt P20933.

b) Presence of AGA in its Active Form:

Enzymatic Studies

Enzymatic studies using specific substrates and inhibitors furthermore made it possible to demonstrate that AGA was present in the SC at least partly in its active, heterodimer or heterotetramer form. The AGA activity was assayed with the substrate Asp-AMC in various SC extracts. An AGA-type activity was demonstrated in protein extracts of reconstructed skin Episkin®, of acetone powder obtained according to the “varnish stripping” technique described by Mehul et al. (2000, The Journal of Biological Chemistry Vol 275, No. 17, 12841-12847) and of plantar stratum corneum. These results are presented in FIG. 1.

Furthermore, specific inhibitors such as L-asparagine used at 1.25 mM inhibit the AGA activity purified from the SC.

Expression (Western)

Two anti-human AGA rabbit antibodies were prepared: they are polyclonal antibodies directed against a particular peptide in the protein sequence respectively on the alpha and beta chains of AGA.

The presence of AGA was demonstrated by immunodetection on various SC extracts after SDS-PAGE separation followed by a transfer onto PVDF membrane. AGA is immunodetected with each antibody diluted 1/1000 and a secondary anti-rabbit antibody coupled to peroxidase is used for revealing with the ECL chemiluminescent reagent.

The antibody directed against the alpha chain of AGA detects a band having a molecular weight of 46-50 kDa on Episkin®-type SC extracts, acetone powder and plantar stratum corneum. A band having a molecular weight of 25 kDa is also detected for an acetone powder extract. The antibody directed against the beta chain of AGA detects a set of bands also around 46-50 kDa.

c) Assay of the activity on various donors:

The AGA activity was assayed directly in the SC from samples of the “blenderms” adhesive type in the forearm, the forehead and the leg of three different donors with the substrate Asp-AMC cited above. Each activity is normalized relative to the quantity of protein of each sample.

The results for the three donors show that AGA can be directly assayed and detected in SC samples and that no great difference in activity is observed according to the donor or the sample area under our experimental conditions.

### Example 2

### Demonstration of the Prodesquamating Effect of AGA

An SC acetone powder obtained by the “varnish stripping” technique as described by Mehul B. et al. (2000, The Journal of Biological Chemistry, Vol. 275, No. 17, 12841-12847) is incubated with a semi-purified AGA extract in the presence or otherwise of 5 mM L-asparagine at the rate of 100 μl of incubation solution per mg of acetone powder. The incubations are carried out in 0.1 M Tris-HCl buffer, pH 8, at 37° C., for 8 hours, with stirring. A test t0 is made as well as a control: buffer alone.

The soluble proteins of each sample are then extracted under denaturing conditions (SDS, DTT and boiling). The total proteins are assayed according to the Bradford method and the concentrations are aligned at 0.6 mg/ml in Laemmli extraction buffer.

An SDS-PAGE separation is then carried out, followed by a transfer onto PVDF membrane marketed by Millipore. The corneodesmosin is immunodetected with the monoclonal antibody G3619 described in Serre G. et al. (1991, J. Invest. Dermatol., 97, 1061-1072) diluted 1/12500. An anti-mouse secondary antibody coupled to peroxidase is used as well as the ECL chemiluminescent reagent marketed by Amersham Biosciences for the revealing of the blots.

Each blot is then stained with a 0.03% amido black solution (SIGMA) in order to normalize the quantity of corneodesmosin relative to a quantification of the keratins.

FIG. 2 presents the variation of the corneodesmosin signal after incubation of various quantities of semi-purified AGA with an acetone powder for 8 hours.

A decrease in the quantity of corneodesmosin is observed after incubation with increasing quantities of AGA.

The presence of L-asparagine at 5 mM inhibits this effect for the 10 μl of AGA condition.

These results therefore make it possible to demonstrate the prodesquamating effect of AGA via the elimination of the corneocytes.

### Example 3

### Use in a Cosmetic Composition—Examples of Formulation

Composition 1: Face Milk

| | | |
|---|---|---|
| Liquid paraffin | 7.0 | g |
| AGA | 0.001 | g |
| Glyceryl monostearate, polyethylene | 3.0 | g |
| glycol stearate (100 EO) | | |
| Carboxyvinyl polymer | 0.4 | g |
| Stearyl alcohol | 0.7 | g |
| Soyabean proteins | 3.0 | g |
| NaOH | 0.4 | g |
| Preservative qs | | |
| Water qs | 100 | g |

This composition is provided in the form of a face milk which has good cosmetic properties and which is smooth and comfortable to use. The pH of the composition is about 6.

Composition 2: Lotion

| | |
|---|---|
| AGA | 0.01 g |
| 2-Ethylhexyl palmitate | 10.0 g |
| Cyclopentadimethylsiloxane | 20.0 g |
| Butylene glycol | 5.0 g |
| Preservative qs | |
| Water qs | 100 g |

This lotion, which contains no surfactant, promotes desquamation of the skin.

Composition 3: Milk

| | | |
|---|---|---|
| Octyl palmitate | 35.0 | g |
| Glycerin | 2.0 | g |
| AGA | 0.1 | g |
| C₁₀-C₃₀ acrylate/alkyl acrylate crosslinked polymer | 0.1 | g |
| Triethanolamine | 0.1 | g |
| Wheat amino acids | 1.0 | g |
| Precursor vit C (bioconvertible molecule) | 0.5 | g |
| Preservative qs | | |
| Water qs | 100 | g |

The milk obtained possesses good cosmetic properties.

Composition 4: Face Gel

| | |
|---|---|
| Glycerin | 10.0 g |
| Disodium cocoamphodiacetate | 1.0 g |
| L-Asparagine (AGA inhibitor) | 0.1 g |
| Preservative qs | |
| Water qs | 100 g |

The gel obtained possesses good cosmetic properties.

Composition 5: Water-based Cleansing Gel

| | | |
|---|---|---|
| Butylene glycol | 7.0 | g |
| Sodium lauroyl sarcosinate | 4.0 | g |
| Amidase (family of hydrolases with amidase activity) | 0.1 | g |
| Triethanolamine | 0.8 | g |
| Carbomer | 0.5 | g |
| Preservative qs | | |
| Water qs | 100 | g |

The gel obtained possesses good cosmetic properties.

Each patent, patent application, publication and literature article/report cited or indicated herein is hereby expressly incorporated by reference.

While the invention has been described in terms of various specific and preferred embodiments, the skilled artisan will appreciate that various modifications, substitutions, omissions, and changes may be made without departing from the spirit thereof. Accordingly, it is intended that the scope of the present invention be limited solely by the scope of the following claims, including equivalents thereof.

What is claimed is:

1. A topically applicable cosmetic/dermatological composition comprising (i) at least one hydrolase polypeptide having amidase activity, or precursor thereof, and (ii) at least one product capable of modulating the activity of said at least one hydrolase polypeptide (i), formulated into (iii) a topically applicable, physiologically acceptable medium therefor.

2. The cosmetic/dermatological composition as defined by claim 1, said at least one hydrolase polypeptide having amidase activity comprising a polypeptide having aspartylglucosaminidase activity, or precursor thereof.

3. The cosmetic/dermatological composition as defined by claim 2, said polypeptide having aspartylglucosaminidase activity being selected from the group consisting of:
(a) a polypeptide of human origin or a homologue of said polypeptide having aspartylglucosaminidase activity;
(b) an enzymatic or biomimetic analogue of said polypeptide (a), having aspartylglucosaminidase activity;
(c) a fragment of said polypeptide (a), said fragment having aspartylglucosaminidase activity;
(d) a polypeptide or an enzymatic analogue or fragment of polypeptide (a), (b) or (c) in an active form of the heterodimer or heterotetramer type; and
(e) a modified polypeptide or a modified enzymatic analogue or a modified polypeptide fragment (a), (b) or (c).

4. The cosmetic/dermatological composition as defined by claim 3, comprising a polypeptide of human origin having aspartylglucosaminidase activity, isolated and purified from the stratum corneum of the human epidermis and having an apparent molecular mass ranging from 10 to 50 kD.

5. The cosmetic/dermatological composition as defined by claim 4, said apparent molecular mass ranging from 15 to 48 kD.

6. The cosmetic/dermatological composition as defined by claim 5, said apparent molecular mass ranging from 15 to 24 kD.

7. The cosmetic/dermatological composition as defined by claim 1, said at least one hydrolase polypeptide having amidase activity comprising a polypeptide of natural origin.

8. The cosmetic/dermatological composition as defined by claim 1, said at least one hydrolase polypeptide having amidase activity comprising a recombinant polypeptide.

9. The cosmetic/dermatological composition as defined by claim 1, said at least one hydrolase polypeptide having amidase activity comprising from 10⁻⁶ to 5% by weight thereof.

10. The cosmetic/dermatological composition as defined by claim 1, said at least one hydrolase polypeptide having amidase activity comprising from 10⁻⁴ to 1% by weight thereof.

11. The cosmetic/dermatological composition as defined by claim 1, said at least one hydrolase polypeptide having amidase activity comprising from 0.001% to 0.1%.

12. The cosmetic/dermatological composition as defined by claim 1, said at least one product capable of modulating the activity of said at least one hydrolase polypeptide comprising an activator that stimulates the activity of said hydrolase polypeptide.

13. The cosmetic/dermatological composition as defined by claim 12, said activator comprising from 0.01% to 50% by weight thereof.

14. The cosmetic/dermatological composition as defined by claim 12, said activator comprising from 0.1% to 1% by weight thereof.

15. A topically applicable cosmetic/dermatological composition comprising (i) at least one hydrolase polypeptide having amidase activity, or precursor thereof, and (ii) at least one activator of said at least one hydrolase polypeptide (i), formulated into (iii) a topically applicable, physiologically acceptable medium therefor.

16. The cosmetic/dermatological composition as defined by claim 15, further comprising at least one other desquamating agent.

17. The cosmetic/dermatological composition as defined by claim 16, further comprising at least one β-hydroxy acid; α-hydroxy acid; α- or β-keto acid; urea; gentisic acid; oligofucoses; cinnamic acid; Saphora japonica extract; resveratrol or derivative thereof; glycosidase, stratum corneum chymotryptic enzyme (SCCE) or other serine or cysteine protease; chelating agent; aminosulphonic comnpound; sugar derivative; reducing agent and/or retinoid.

18. The cosmetic/dermatological composition as defined by claim 1, said at least one product capable of modulating the activity of said at least one hydrolase polypeptide comprising an inhibitor of the activity of said hydrolase polypeptide.

19. The cosmetic/dermatological composition as defined by claim 18, said inhibitor comprising from 10⁻⁶% to 5% by weight thereof.

20. The cosmetic/dermatological composition as defined by claim 18, said inhibitor comprising from 0.1% to 1% by weight thereof.

21. The cosmetic/dermatological composition as defined by claim 1, further comprising at least one moisturizing agent; propigmenting agent; agent stimulating the synthesis of dermal or epidermal macromolecules and/or preventing the degradation thereof; agent stimulating the proliferation or differentiation of keratinocytes; relaxing agent; antipollution and/or anti-free radical agent; UV-screening agent; permeating agent; cicatrizing agent; and mixtures thereof.

22. A regime or regimen for the treatment of dry skin, for combating skin desquamation and/or for promoting cell renewal of the epidermis and/or for promoting hydration of the skin and/or for promoting cell proliferation and/or differentiation in the skin, comprising topically applying onto the skin, mucous membranes and/or keratinous fibers of an individual in need of such treatment, thus effective amounts of (i) at least one hydrolase polypeptide having amidase activity, or precursor thereof, and (ii) at least one product capable of modulating the activity of said at least one hydrolase polypeptide (i).

23. A regime or regimen for the treatment of sensitive and/or aged skin, for enhancing the barrier effect thereof, comprising topically applying onto the skin, mucous membranes and/or keratinous fibers of an individual in need of such treatment, thus effective amounts of (i) at least one hydrolase polypeptide having amidase activity, or precursor thereof, and (ii) at least one inhibitor of the activity of said hydrolase polypeptide.

24. A regime or regimen for the treatment of hyperkeratosis, xerosis, ichtyosis, psoriasis or reactive keratosis, comprising topically applying onto the skin, mucous membranes and/or keratinous fibers of an individual in need of such treatment, thus effective amounts of (i) at least one hydrolase polypeptide having amidase activity, or precursor thereof, and (ii) at least one activator of said at least one hydrolase polypeptide (i).

25. A regime or regimen for promoting cicatrization, comprising topically applying onto the skin, mucous membranes and/or keratinous fibers of an individual in need of such treatment, thus effective amounts of (i) at least one hydrolase polypeptide having amidase activity, or precursor thereof, and (ii) at least one activator of said at least one hydrolase polypeptide (i).

26. A regime or regimen for the treatment of atopic dermatitis, comprising topically applying onto the skin, mucous membranes and/or keratinous fibers of an individual in need of such treatment, a thus effective amount of at least one inhibitor of a hydrolase polypeptide having amidase activity.

27. A regime or regimen for promoting desquamation and/or hydration of the skin and/or cell renewal in the skin and/or cell proliferation and/or differentiation in the skin of an individual in need of such treatment, comprising topically applying thereon thus effective amounts of (i) at least one hydrolase polypeptide having amidase activity, or precursor thereof, and (ii) at least one activator of said at least one hydrolase polypeptide (i).

28. A regime or regimen for facilitating the penetration into the skin of a cosmetic/dermatological active agent, comprising, therewith, topically applying thereon thus effective amounts of (i) at least one hydrolase polypeptide having amidase activity, or precursor thereof, and (ii) at least one activator of said at least one hydrolase polypeptide (i).

29. A regime or regimen for combating bacterial adhesion to the skin, comprising topically applying onto the skin of an individual in need of such treatment, thus effective amounts of (i) at least one hydrolase polypeptide having amidase activity, or precursor thereof, and (ii) at least one activator of said at least one hydrolase polypeptide (i).

30. A package confining (i) at least one hydrolase polypeptide having amidase activity, or precursor thereof, and (ii) at least one product capable of modulating the activity of said at least one hydrolase polypeptide (i), each of said hydrolase polypeptide (i) and modulator (ii) being packaged in separate compartments thereof, each not physically in contact with the other.

31. The package as defined by claim 30, said modulator (ii) comprising an activator of said at least one hydrolase polypeptide.
